(19) United States
(12) Patent Application Publication
(10) Pub. No.: US 2005/0070009 A1
(43) Pub. Date: Mar. 31, 2005

(54) Biopolymer synthesis substrate and method for producing biopolymers

(76) Inventors: Holger Klapproth, Freiburg (DE); Mirko Lehmann, Freiburg (DE); Ingo Freund, Vogtsburg (DE); Joachim Sturken, Jungholtz (FR)

Correspondence Address: Patrick J O'Shea;Gauthier & Connors, Suite 3300, 225 Franklin Street, Boston, MA, 02110, US

(21) Appl. No.: 10/495,845

(22) PCT Filed: Nov. 12, 2002
(86) PCT No.: PCT/EP02/12606

(30) Foreign Application Priority Data
Nov. 16, 2001 (DE) 101 56 467.8

(51) Int. Cl. C12M 1/34; C08G 63/48; C08G 63/91

(52) U.S. Cl. 435/287.2; 525/54.1; 525/54.2

(57) ABSTRACT

The invention concerns a biopolymer synthesis substrate, its use and a method for producing biopolymers

The present invention relates to a substrate for synthesizing organic polymers or biopolymers with a matrix on whose surface the biopolymers are synthesized and an energy source for targeted activation of partial regions of the matrix, wherein the biopolymers are synthesized at the activated partial regions of the matrix. The substrate is used as a sensor chip in a medical instrument, particularly a diagnostic or therapeutic instrument. In addition, the invention relates to a method for synthesizing biopolymers, in which the substrate is used.

Biological systems are based on the interaction of biologically active macromolecules. The precondition for such interactions is activity of the macromolecules, which is determined by their spatial structure. Clarification of the relationship between the spatial structure and the activity of macromolecules is critical when investigating complex biological systems. Clarification of biological interactions makes it possible to understand how cells communicate with other cells in the same group, how enzymes bind and convert their substrate, and how cellular control mechanisms function—or are blocked when cancer occurs. Many biological macromolecules can bind other molecules using their three-dimensional surface structure and a specific electron charge distribution, and interact with them. All molecules with such specificity are known collectively as “receptors.” Examples of such receptors are enzymes that catalyze hydrolysis of a metabolic intermediate, proteins that make it possible for charged molecules to be transported through a biomembrane, glycoproteins that allow contact with other cells, and antibodies that circulate in the blood and detect, bind, and inactivate components of bacteria or viruses, and DNA, the carrier of hereditary information, to which sequence-specific proteins bind and allow their biological use in the cells. Molecules that bind specifically to a receptor are known collectively as ligands; many biological molecules not only bind actively but are in turn bound by other molecules, so that they are both ligands and receptors.

Many test systems (assays) have been developed for investigating the interactions between receptors and ligands to determine their binding affinities and to clarify binding strengths and specificities. In simple biological assays, which are still used today in medical diagnosis, antigenic fragments of bacteria or viruses are fixed on solid surfaces. Next, a test (blood) specimen from a patient is spotted onto the surface, and an interaction between specific antibodies in the (blood) specimen with the antigen fragments can be detected by a detection system. Such an antigen-antibody test is however severely limited by the number of antigen fragments that can be fixed on the microscope slide.

Now that the complete genomic DNA sequences of important model and research organisms such as bacteria (Bacillus subtilis, Escherichia coli), and yeasts (Saccharomyces cerevisiae, Schizosaccharomyces pombe) have been available for several years in databases, it has recently become possible also to sequence the human genome under the Human Genome Project. It is becoming increasingly important to investigate the functions of the individual genes, which have different activities in different tissues and organs. Clarification of differential gene expression is decisive for understanding why cancer appears. Today it is already known that many individuals are at high risk of developing cancer because they have a particular genetic pattern. Although many attempts have been made over a number of years to synthesize artificially the largest possible number of DNA sequences in the smallest possible space so that their interaction with other molecules can be investigated, the ratio of available space to number of possible DNA molecules remains, as formerly, an incompletely solved problem.

Traditional methods are based on automated solid-phase methods to synthesize DNA arrays by sequential addition of active monomers to a growing chain which is bound to an insoluble matrix. Such artificial biological systems are known as “DNA chips.” When DNA chips are made, first the monomers (nucleotides) from which the DNA array is constructed are microdeposited at the points where an oligonucleotide is to be synthesized. Because this method is very cumbersome in practice, it has been superseded by light-directed (i.e., photolithographic) synthesis for making highly dense DNA chips; today this is the method in most-widespread use.

In light-directed DNA chip synthesis, individual masks, known from the semiconductor industry, are used for selective exposure. Thus, the surface of a solid substrate is modified with light-sensitive or “photolabile” protective groups, after which it is exposed through a photolithographic mask placed over it. Exposure leads to selective removal of the protective groups at the exposed spots, causing reactive hydroxyl groups (OH groups) to be released exclusively at the exposed regions. Next, an activated deoxynucleotide, which for its part has an OH group provided with a protective group, is supplied so that coupling of the deoxynucleotide occurs at the previously exposed spots. After an oxidation reaction, the substrate is rinsed and the surface is exposed through a second mask, causing the protective groups to be removed at other spots and activated for another coupling. Then, a second deoxynucleotide, also provided with protected hydroxyl groups, is added. The cycles of exposure to remove protective groups and coupling of the deoxynucleotides is continued until the desired oligonucleotides have been produced on the solid substrate. This makes it possible to produce highly dense DNA arrays (Pease et al., 1994, PNAS, USA, Vol. 91, pp. 5022-5026).

Also, EP 476,014 B1 describes a method for creating polymer libraries on solid substrates, also using photolabile protective groups and splitting them off by appropriate exposure techniques. For each of the monomeric bases—(deoxy)adenine, (deoxy)cytosine, (deoxy)guanine, and (deoxy)thymine—different photolithographic masks must however be present, so that the number of different masks needed is four times the length of the DNA sequences to be synthesized. Compared with synthesis of artificial DNA sequences, mask-based synthesis of peptides is even more cumbersome because 20 natural amino acids are available for building peptides, so that the number of masks is twenty times the length of the peptides. The required set of masks must not only be prepared before synthesis even begins, but must also be precisely aligned after each exposure to prevent incorrect exposures and consequent impurities. U.S. Pat. No. 5,143,854 discloses a light source that makes it possible to move the microscope slide; however the considerable technical complexity makes the masking method unsuitable, especially for small series, as a new set of masks has to be prepared for each new synthesis.

To circumvent the expensive and cumbersome process of mask production, mask-free systems have been developed. WO 99/42813 discloses the building of biopolymers such as DNA arrays or polypeptides in which they are exposed by individual directable micromirrors. The micromirrors form a continuous array composed of electronically directable individual micromirrors (digital mirror devices). A common light source is associated with the micromirror array. The biopolymers located on a slide are activated in specific patterns, and the monomers presented (e.g. the four different bases) become coupled to the directed regions. This process is continued until all the biopolymers are built to the desired length.

The exposure method described uses monomeric building blocks, that initially have reactive groups provided with protective groups, to make location-specific synthesis possible. The action of light removes the photolabile protective groups from the monomeric building blocks, then allows synthesis to take place at the points where the light has acted. Photolabile protective groups are known for example from DE 4444996 Al, which describes nucleotide derivatives with photolabile protective groups for the 5′-OH group in the sugar portion of the bases. When a reactive OH group has been generated, the next monomer can be coupled to the reactive group in the following reaction step. This makes it possible to build any given polymer by alternating removal of the protective group and a coupling reaction.

DE 199 62 803 A1 describes a method using planar substrates for synthesizing a number of different spatially separated polymers at the same time. Several light-emitting diodes (diode array) are used for selective exposure to light. This method uses electrically controllable LEDs for selective removal of protective groups, thus likewise dispensing with expensive masks. The monomers for the biopolymers to be synthesized are arranged in their own device below the optically transparent region. The chemicals required for the synthesis can be provided individually and sequentially in this device. A computer uses an appropriate program to control the individual LEDs in the diode array correlated with the sequential, cyclic provision of the individual monomers. To exclude external interference during exposure, the points where chemical synthesis takes place and the exposure device are kept strictly apart from each other. Thus, like mask technology and exposure by micromirror array, this method suffers above all from blurred exposure due to the spatial separation between synthesis and exposure. Once synthesis is complete, there are no truly discrete regions on the chips but transitions between two or more defined products. The blurred transitions are brought about in particular by diffraction of light on the masks and the fuzziness of images.

Accordingly, the goal of the present invention is to provide a substrate for synthesizing biopolymers that avoids both the diffraction effect of light and blurred imaging whereby exact pre-defined regions on the substrate where biopolymer synthesis will take place can be selected. Nonspecific transitions due to absent light, additional light, or edge-lighting are to be avoided.

This goal is achieved according to the invention by a substrate for the synthesis of biopolymers having a matrix on whose surface the biopolymers are synthesized and an energy source for targeted activation of partial regions of the matrix, with the biopolymers being synthesized on the activated partial regions of the matrix and the matrix and the energy source forming a single unit. The matrix plus energy source unit makes it possible effectively to impede the diffusion and diffraction effects of light. In this way, arrays of biopolymers such as oligonucleotides or peptides can be synthesized over the entire surface of the substrate. The undesirable transitions between two or more defined products are completely absent, so that special tests for detecting “false” biopolymers produced by improper exposure are unnecessary. Thus, a larger number of biopolymers can be synthesized on the substrate for the same surface area.

Other subjects of the present invention are a sensor chip and a medical instrument, particularly a diagnostic or therapeutic instrument, containing the substrate.

Moreover, the present invention relates to a method for synthesizing biopolymers in which the substrate according to the invention is used. Other steps of the method according to the invention relate to targeted activation of partial regions of the matrix by splitting off the protective groups in selected partial regions, supplying biomonomers that also have protective groups, and interaction of the biomonomers with the target-activated partial regions of the matrix. The energy for targeted activation of partial regions of the matrix by which the protective groups are split off in the selected partial regions is emitted within the substrate.

With the substrate and the method according to the invention, no type of imaging optics is required, biopolymer production requires less-cumbersome synthesis technology, and at the same time synthesis quality is considerably improved because nonspecific transitions between individual biopolymers, representing contamination of the synthetic end product, are avoided. In this way, biopolymer arrays can be produced quickly, individually, and flexibly. Moreover, simple targeted, selective synthesis of biopolymers is possible with low cost and time expenditure.

Terms used to describe and present the invention will now be defined.

1. Ligand

A ligand is a molecule that is recognized by a certain receptor. Ligands are both naturally occurring and artificial substances. Examples of ligands are agonists and antagonists of cell membrane receptors, toxins, viral and bacterial epitopes*, hormones (opiates*, steroids, etc.), peptides, enzymes, enzyme substrates, cofactors, medicinal agents, sugar molecules, lecithin, oligonucleotides, nucleic acids, oligosaccharides, proteins, peptides, and lipids.
German has “eptitopes” (Eptitope) and “optiates” (Optiate), presumably in error. Translator.

2. Receptor

A receptor is a molecule with a binding affinity for a particular ligand. Receptors are both naturally occurring and artificial substances. They may also be present in their natural state or as aggregates with other molecules. Receptors bind to the ligands, covalently or noncovalently, directly or indirectly through specific binding substances or binding molecules. Examples of receptors are antibodies, particularly monoclonal and polyclonal antibodies, antisera, cell membrane receptors, polynucleotides, nucleic acids, cofactors, lecithin, sugar molecules, polysaccharides, cells, cell membranes, and organelles. With the corresponding ligands, receptors form a “ligand-receptor complex” due to their molecular recognition.

3. Organic Polymers

Organic polymers are formed from small organic compounds (monomers) by reacting with each other or with other small organic compounds by the reaction process known as polymerization, the resulting product (a polymer) being a compound with a relatively high molecular weight. Examples of organic polymers are polymers of alkenes such as polyethylene, polypropylene, or modified polymers such as polyvinyl chloride, Teflon, polystyrene, and polyamides (Nylon).

4. Biomonomer

A biomonomer is a single building block or a set or group of individual small building blocks which can bond together thus forming a biopolymer. Examples of biomonomers are the 20 naturally occurring L-amino acids, D-amino acids, artificially synthesized amino acids, nucleotides, nucleosides, sugar molecules such as pentoses or hexoses, as well as short-chain peptides such as tetramers or pentamers. The term “biomonomer” as used in the context of the present invention relates to all building blocks used for synthesis of a biopolymer. If, instead of individual amino acids, short peptide sequences such as tetramers, pentamers, or hexamers are used as the biopolymer for building a protein, the building blocks consisting of four, five, or six amino acids are also termed “biomonomers.”

5. Biopolymer

A biopolymer is a product synthesized from biomonomers, regardless of its length and individual components. If three different amino acids are used as biomonomers, the resulting trimer is termed a “biopolymer.” A biopolymer can be constructed from a number of the same biomonomers or from different biomonomers.

6. Protective Groups

A protective group is any material bound bonded to a monomer and used to modify the monomer. The protective group can be selectively split off by exposure to an energy source such as light. Splitting off the protective group lays bare a reactive group such as a hydroxyl group. Examples of protective groups are nitroveratryloxycarbonyl, nitrobenzyloxycarbonyl, dimethyldimethoxybenzoyloxycarbonyl, 5-bromo-7-nitroindolinyl, hydroxy-α-methylcinnamoyl, 2-oxymethylenanthraquinone, and p-nitrophenylethoxycarbonyl [groups*].
* Word added by translator.

7. Analogs and/or Derivatives

Analogs and/or derivatives are understood to be all naturally occurring and artificially synthesized modifications of biomonomers and biopolymers. Known analogs of nucleic acids are for example PNA or LNA.

Advantageous embodiments of the substrate according to the invention and the method will now be described.

A three-dimensional matrix (3D matrix) composed of a polymer layer is used as the matrix for efficient synthesis of biopolymers. Either a crosslinked or a non-crosslinked polymer can be used, a crosslinked polymer with a low degree of crosslinking being especially suitable. A suitable polymer layer has a plurality of individual polymer chains bound to a solid surface. There is preferably a covalent bond between the polymer chains and the solid surface. Not only linear polymers but also branched polymers may be used. Due to the large surface area of the three-dimensional polymer layer, there are ample numbers of places for biopolymer synthesis to begin. Examples of polymers suitable for building the 3D matrix may also be taken from EP 1,035,218 A1.

If a thin polymer layer is used as the matrix, particularly one with a thickness of 30 to 3000 nm, synthesis of the biopolymers is not affected by the three-dimensional polymer layer. It has proved especially advantageous to use as a matrix at least one polymer layer that is swellable in partial regions. Swellability in water is ensured by components such as acrylic acid, methacrylic acid, dimethylacrylamide, or vinylpyrrolidone. In its swollen state the polymer layer preferably has a thickness of 50 to 500 nm.

The three-dimensional polymer layer also has reactive starter groups. The reactive starter groups are preferably hydroxyl groups (OH groups) that are directly bonded to the three-dimensional polymer layer. Alternatively, the reactive starter groups may also be connected to the polymer layer through other functional groups, particularly low-molecular-weight chemical compounds. A covalent bond that ensures a durable connection of the reactive starter group is particularly suitable for the connection between the polymer layer and the reactive starter group.

The reactive starter groups are protected by protective groups. As long as the protective group is connected to or with the starter group, the starter group is inactive. It does not become active until the protective group is split off.

Biopolymers that can be synthesized with the aid of the substrate are receptors or ligands, nucleic acids, oligonucleotides, proteins, peptides, polysaccharides, lipids, and their derivatives or analogs. In synthesizing longer biopolymers, it has proved useful to employ partial structures of the biopolymer as the monomer. Tetramers are particularly suitable for this purpose. The long-chain monomers are assembled into biopolymers. This reduces the number of reaction steps, substantially improving the purity and yield of the finished synthesized biopolymers. Thus, by using 256 tetrameric oligonucleotides, any dodecamer* can be made in only five coupling steps, whereas formerly 20 coupling steps were required. The use of tetramers as biomonomers is particularly advantageous for synthesis of high-quality homologous DNA sequences, because only a few oligomers are needed as biomonomers. By using oligomers as biomonomers, in particular polymer sequences may be made which formerly could not be made with adequate quality and yield in a traditional solid-phase synthesis because of their length or the number of coupling steps required.
* Sic—this presumably should mean biopolymers containing 20 biomonomers, but “dodecamer” means containing 12 biomonomers. Translator.

Electrically controllable light emitting diodes (LEDs) or laser diodes (LDs) are suitable for the energy source needed for splitting off the protective groups, and for the associated activation of the reactive OH groups. If LEDs are used, it is advantageous for them to produce high-energy radiation in the UV range. UV light has proved especially suitable for splitting off protective groups. Examples of UV LEDs are particular compounds from III-V semiconductors. Thus, for example, a gallium nitride (GaN) LED emits light with a wavelength of 380 nm (see for example Rep. Prog. Phys. 61 (1998) 1-75; Group III nitride semiconductors for short wavelength light-emitting devices). Moreover, when AlGaN compounds are used, wavelengths of between 200 and 360 nm can be reached. It is particularly preferable to integrate several LEDs and/or the signal processing/control monolithically in one substrate.

In another preferred embodiment, the substrate does not consist only of an array of light-emitting diodes, but also has detectors. By connecting synthesis of the biomolecules with a detector, in the form of a camera, external detection becomes unnecessary. The detector itself detects interactions between the biopolymers synthesized on the surface of the matrix and test specimens. “Interactions” are understood to be all interactions between biopolymers and other molecules, particularly formation of covalent bonds, ionic interactions, van der Waals forces, and hydrogen bridge bonds. Candidates for test specimens are all types of biological or artificial samples, particularly blood samples, patient material, smears, nose and throat swabs, epidermal scales, and saliva samples. These specimens have receptors or ligands that interact with the biopolymers (which themselves are ligands or receptors). If for example a single-strand nucleic acid (single-strand DNA, RNA, or single-strand cDNA) is used as the biopolymer, the complementary single strand from the specimen can be detected by hybridizing the two complementary strands. Such hybridization represents a receptor/ligand reaction according to the invention. Interactions between biopolymers and specimens are detected by chemical or biochemical reactions. Luminescence is particularly suitable for this purpose. However, other detection methods using streptavidin or radioactive labeling or label-free methods may be used (see for example Souteyrand, E., Cloarec, J. P., Martin, J. R., Wilson, C., Lawrence, I., Mikkelsen, S., Lawrence, M. F., 1997. Direct detection of the hybridization of synthetic homo-oligomer DNA sequences by field effect. J. Phys. Chem. B, 1001, 2980 as well as DE 4318519 C2 “Electrochemical Sensor”). Particularly preferably, these sensors and their signal processing are integrated monolithically in the substrate.

It has proved especially advantageous for the matrix plus energy source unit to be highly compact and for the average distance between the matrix and energy source to be less than 10 microns.

Because of this short distance, biopolymer synthesis takes place in the immediate vicinity of the energy source. The starter groups and the growing biopolymers are thus spatially close to the LEDs, which emit the UV light needed for splitting off the protective groups. Because of the spatial proximity of the starter groups and the LEDs, light diffusion and refraction effects are efficiently avoided. The short distance between the matrix and the energy source may be achieved in particular by direct coating of the UV-emitting LEDs with the matrix.

Glycidoxypropyltrimethoxysilane is a suitable material for the matrix in such a case, and can be applied to the LEDs in a coating process.

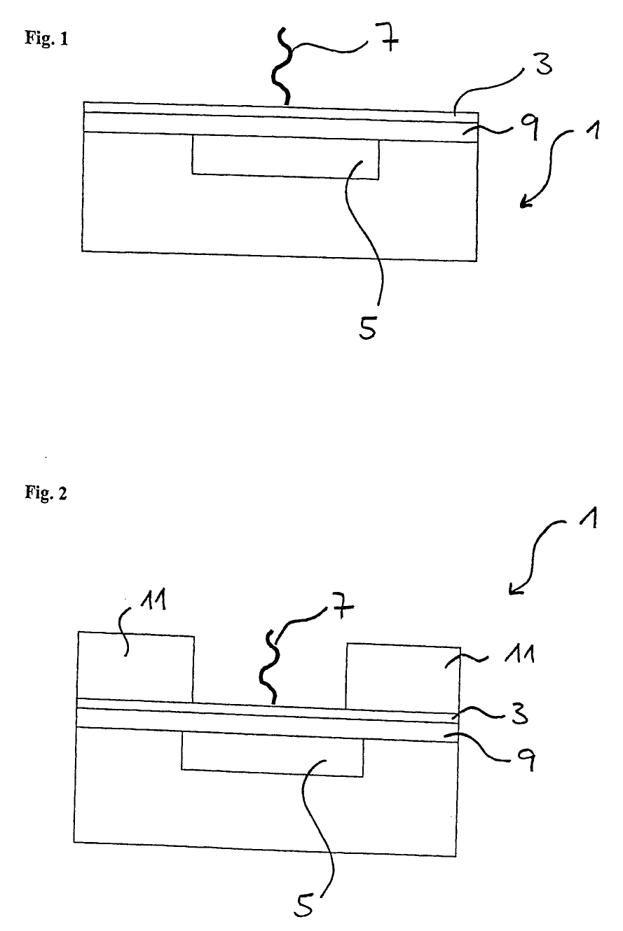

This embodiment of the invention is also illustrated in FIG. 1. The substrate 1 has an energy source 5 and a matrix 3 on the surface of which the biopolymers 7 are synthesized. The energy source 5 is in the form of an LED. The LED has a pn transition with an insulator 9. Photons are generated at this pn transition which is thus the energy source 5. The average distance between matrix 3 and energy source 5 of less than 10 microns is determined by insulator 9.

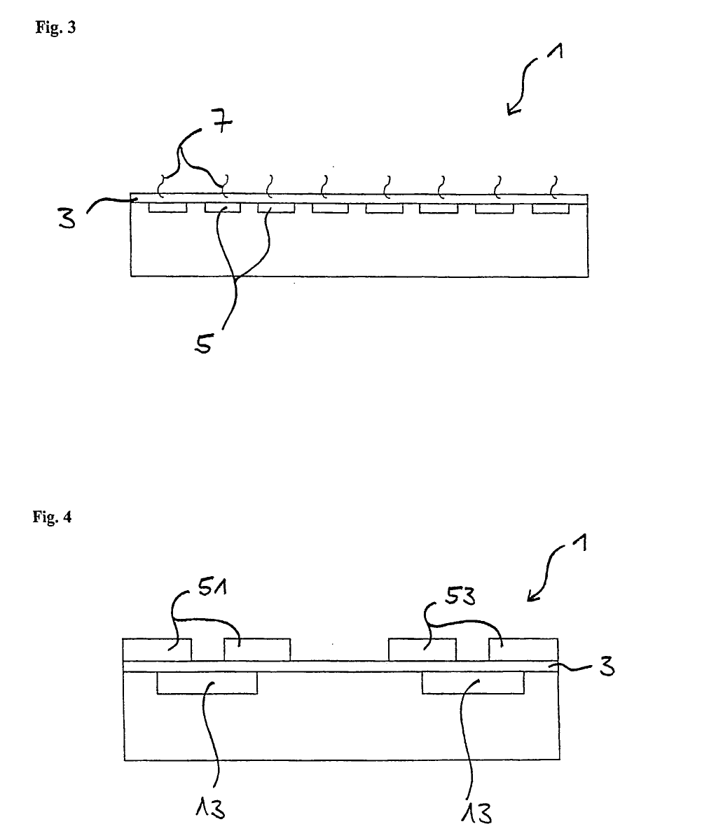

An alternative embodiment of the present invention is shown in FIG. 3. The substrate 1 once again has the matrix 3 and energy source 5. Energy source 5 in this embodiment consists of a plurality of LEDs, so that a plurality of biopolymers 7 can be synthesized simultaneously on matrix 3 (array arrangement).

Another subject of the present invention is a method for synthesizing biopolymers in which the substrate according to the invention is used. This substrate is prepared, then partial regions of the matrix are target-activated by splitting off the protective groups in the selected partial regions. Then biomonomers, which also have protective groups, are added and these biomonomers interact with the target-activated partial regions of the matrix. The sequential cycles of targeted activation of partial regions of the matrix, the provision of protected biomonomers, and the interaction of the biomonomers with the target-activated partial regions of the matrix are repeated until the desired biopolymer is produced. In the context of the method according to the invention, the energy needed for targeted activation of partial regions is emitted inside the substrate. The compact unit consisting of an energy source and a matrix is particularly suitable for this purpose.

In order to synthesize a plurality of different polymers at the same time, the partial regions are selected and activated with the aid of a computer.

To split off the protective groups for activation of the reactive starter groups, which are preferably OH groups, a local pH change can be used. Here, the substrate according to the invention has an electrode structure as an energy source. By applying voltages and currents, locally very substantial pH changes can be produced. Differences in pH of approximately 5 can be achieved between the individual electrodes. Protective groups can be removed at a pH of 2 for example. By electrolysis of water at the electrode, a basic environment is produced when a negative voltage is applied to the electrode. An acidic environment can be produced by applying a positive voltage to the electrodes. Chemically, the following reactions take place with electrolysis of water at the electrode:
2H₂O→4H⁺+4e⁻+O₂ (with positive voltages); and
2H₂O+2e⁻→2OH⁻+H₂ (with negative voltages).

With a neutral pH of 7, the reactive starter groups are protected by intact protective groups. The protective groups are selectively removed by a positive voltage at microelectrodes, which because of the energy source plus matrix unit are in the immediate vicinity of the protective groups, because a local drop in pH is produced by the positive voltage. It is also particularly advantageous for a pH measuring device (pH-ISFET) to be located in the immediate vicinity of the electrodes, with the aid of which not only can the existing pH be detected but also the electrode can be electrically controlled, which induces a local change in pH.

This arrangement is additionally illustrated in FIG. 4. The substrate 1 has a matrix 3 and an energy source, in the form of electrodes 51 and 53. Shown at the left in FIG. 4 are electrodes 51 to which a voltage/current is applied. The pH is 7. Electrodes 53 are shown at the right, to which electrodes no voltage/current is applied. The pH is 2. The detector 13, in the form of a pH-ISFET, serves to detect the local pH generated by the electrode.

Any biomonomers may be used for making biopolymers in the context of the present invention. Nucleotides, oligonucleotides, in particular tetramers, amino acids, peptides, saccharides, particularly mono- and disaccharides and/or their derivatives or analogs are particularly suitable.

The biomonomers may be derived for various screening processes from a cDNA, RNA, genomic DNA library and/or a peptide library.

It has proved especially advantageous to supply the biomonomers in a feeder. Other reaction steps advantageous for building the biopolymers, such as washing steps, may also take place in the feeder. The surface is wetted or coated with the appropriate reaction solutions, particularly the biomonomers, in the feeder. The feeder may be in the form of a microfluidic cuvette or chamber. To avoid impurities, at least one feeder for the reaction solutions or biomonomers as well as at least one removal device spatially separated from the feeder have proven advantageous. The surface of the microfluidic cuvette simultaneously serves as a light trap to prevent improper exposure of the substrate due to scattered light. It is especially advantageous if channels are provided either monolithically or hybrid-fashion on the chip to shield it from scattered light from other LEDs or the like.

This embodiment is also illustrated in FIG. 2. The substrate 1 has a matrix 3, an energy source 5, and an insulator 9. Synthesis of the biopolymers 7 takes place in depressions in matrix 3, in the form of channels 11.

Moreover, it has proved advantageous to use electric fields to bring charged biomonomers to the activated partial regions of the matrix. The partial regions of the matrix are selected by applying at least one electric field. When this is done, the protective groups are completely removed, after which the charged biomonomers are added sequentially. The charged biomonomers are attracted by the electric field to the selected spots while they are electrically repelled from the undesired spots. In this way, a biopolymer can be built by sequential addition of the four different bases (adenine, thymine, cytosine, and guanine) modified with protective groups. By appropriate attraction or repulsion of the charged nucleotides in the electric field, they are brought to the desired position on the matrix. After removal of the protective groups from the bonded nucleotides, an electric field is again applied and new nucleotides are added until the desired length of the biopolymers is reached.

If, in the context of the method according to the invention, a substrate with a detector as described above is used, the method according to the invention may have additional steps. The synthesized biopolymers react with test specimens and the receptor-ligand complexes are detected by a biochemical reaction, particularly by bioluminescence and/or chemoluminescence.

Another subject of the present invention is a sensor chip that has a substrate according to the invention. Moreover, the invention relates to a medical instrument, particularly a diagnostic or therapeutic instrument, which also includes the substrate according to the invention. If for example receptive elements are used on the sensor chip, particularly on a DNA sensor chip, the invention permits portable DNA analysis technology using probe molecules that proved to be usable in the course of the prior analyses. Such a sensor chip or medical instrument can be used in a disease outbreak to identify the bacterial or viral pathogen on the spot, and distinguish between infected and uninfected individuals. Moreover, by building in logic switching elements, complex issues of DNA analysis, such as the biopolymer design of the instrument, can be processed by the instrument itself. The medical instrument can thus, given the issues described above, develop the appropriate sensor chip itself, after which it synthesizes the needed biopolymers and then evaluates the results.

FIGS. 1 to 4 (FIGS. 1-4) will further explain the invention.

FIG. 1 is a first embodiment of the invention in which the matrix plus energy source unit is shown simply;

FIG. 2 is a second embodiment of the invention in which the biopolymers are produced vin depressions (channels) in the matrix;

FIG. 3 is another embodiment of the invention in which a plurality of LEDs is used for simultaneous synthesis of a plurality of biopolymers; and

FIG. 4 is an alternative embodiment of the invention in which removal of the protective groups by local pH changes is shown schematically.

The following example will explain the invention in greater detail and represents a simple embodiment example.

### EXAMPLE

1. Coating the Sensor

A CMOS sensor is coated with silane for 2 hours by dipping it in a solution of 1% glycidoxypropyltrimethyoxysilane (=1% GOPS) and 0.1% triethylamine in toluene. The chip is then allowed to drain and is fixed at 120° C. for about two hours in a drying oven. Until it is coated with protective groups, the prepared chip can be stored protected from moisture.

2. Functionalizing the Silane-Coated Chip with Hydroxyl Groups

The pretreated chip is incubated for 1 hour in hot (70° C.) ethylene glycol that has a catalytic quantity of concentrated sulfuric acid. The chip is then washed in ethanol and dried. After this treatment, the chip has a hydroxy-functionalized surface, and the OH groups are now reactive starter groups.

3. Application of Protective Groups to Starter Groups

The starter groups on the surface of the chip are protected by applying a pNPEOC group. Namely, the pretreated chip is incubated in a solution of 2-(5-methyoxy-2-nitrophenyl)ethoxycarbonyl chloride in dichloromethane for 4 hours at −15° C. protected from light. The protective group has the following chemical formula:

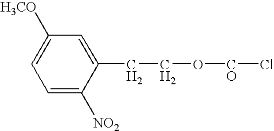

The chip is then rinsed in cold dichloromethane. Until the chip is used, it is kept dry and protected from light.

4. Synthesis of Biopolymers on the Pretreated Chip, using UV Light

The pretreated chip is placed in a cabinet and the protective groups are split off at the predetermined regions by activation with UV LEDs for 2 minutes. The chip is then rinsed in anhydrous acetonitrile and incubated with a first nucleotide dissolved in acetonitrile. Commercially available nucleotides with pNEPOC protective groups are used. The chip is then washed again with acetonitrile and more or other protective groups are removed by selective exposure. In this manner, all positions at which adenine-, guanine-, cytosine-, or thymidine-modified nucleotides are to be incorporated are selectively deprotected. After coupling of all four nucleotides, there is once again a pNPEOC protective layer at all the positions on the chip, and the next nucleotide layer can then be added by targeted deprotection and addition of the nucleotides.

Examples of commercially available thymidine or cytosine derivatives:
5′-O-(2-(2-chloro-6-nitrophenyl)ethyoxycarbonylthymidine-3′-O((β-cyanoethoxy)(N,N-diisopropylamino)phosphoramidite)
5′-O-(2-(2-chloro-6-nitrophenyl)ethoxycarbonyl)-N-4-(4-nitrophenyl)ethoxycarbonyl)-2′-desoxycytidine-3′-O-((β-cyanoethoxy)(N,N-diisopropylamino)phosphoramidite)
   List of Reference Numbers
1 substrate
2 matrix
3 energy source
51 electrodes—pH=7
53 electrodes—pH=2
7 biopolymer
9 insulator
11 channels
13 detector

1. Substrate for synthesis of organic polymers, comprising biopolymers with a matrix on whose surface the biopolymers are synthesized and an energy source for targeted activation of partial regions of the matrix, wherein the biopolymers are synthesized at the activated partial regions of the matrix, wherein the matrix and the energy source form a single unit.

2. The substrate of claim 1, wherein the matrix comprises a swellable three-dimensional polymer layer with reactive starter groups.

3. The substrate of claim 2, wherein the polymer layer comprises crosslinked polymers with a low degree of crosslinking.

4. The substrate of claim 2, wherein, the polymer layer has a thickness of between 30 and 3000 nm.

5. The substrate of claim 4, wherein the swollen polymer layer has a thickness of 50 to 500 nm.

6. The substrate of claim 2, wherein, the reactive starter groups are connected covalently, directly or through other functional groups, with the polymer layer.

7. The substrate of claim 2, wherein, the reactive starter groups are OH groups.

8. The substrate of claim 2, wherein, the reactive starter groups are protected by unreactive protective groups.

9. The substrate of claim 1, wherein, the biopolymers are receptors, ligands, nucleic acids, oligonucleotides, proteins, peptides, polysaccharides, lipids, and/or their derivatives or analogs.

10. The substrate of claim 1, wherein, the energy source comprises Fat least one electrically controllable light-emitting diode (LED) or at least one laser diode (LD).

11. The substrate of claim 10, wherein, the light-emitting diodes emit high-energy radiation in the UV range.

12. The substrate of claim 1, wherein, the substrate also has at least one detector.

13. The substrate of claim 12, wherein, the detector is in the form of a camera.

14. The substrate of claim 12, wherein, the detector detects interactions between the biopolymers synthesized on the surface of the matrix, and test specimens.

15. The substrate of claim 14, wherein, the test specimens have ligands, receptors, proteins, antibodies, peptides, nucleic acids, and/or their derivatives or analogs.

16. The substrate of claim 14, wherein, the interactions are detected by a (bio)chemical reaction, comprising the group of luminescence, by radioactive and/or non-radioactive labeling, or biotinylization reactions with streptavidin.

17. The substrate of claim 1, wherein, the average distance between biomolecules immobilized in the matrix and the energy source is less than about 10 microns.

18. The substrate of claim 11, wherein the UV-emitting LEDs are coated with the matrix, in particular with a glycidoxypropyltrimethoxysilane.

19. The substrate of claim 1, wherein the energy source is integrated in monolithic fashion into the substrate.

20. A method for synthesizing biopolymers, comprising the following steps:
(a) preparing a substrate:
(b) targeted activation of partial regions of the matrix by splitting off the protective groups in the selected partial regions;
(c) adding biomonomers, which in their turn have protective groups;
(d) causing the biomonomers to interact with the target-activated partial regions of the matrix from step (b);
(e) repeat steps (b) to (d) if appropriate; wherein
the energy for activation in step (b) is emitted inside the substrate.

21. The method of claim 20, wherein, the partial regions are selected and activated by computer.

22. The method of claim 20, wherein, the protective groups are split off by local changes in pH.

23. The method of claim 22, wherein, the local pH value is changed by at least one electrode, to which a positive voltage is applied.

24. The method of claim 20, wherein the biomonomers are selected from the group comprising nucleotides, oligonucleotides, in particular tetramers, amino acids, peptides, saccharides, or mono- and disaccharides, and/or their derivatives or analogs.

25. The method of claim 20, wherein the biomonomers are derived from a cNDA, RNA, genomic DNA library, and/or a peptide library.

26. The method of claim 20, wherein the biomonomers are supplied in a feeder.

27. The method of claim 26, wherein the feeder is in the form of a microfluidic cuvette or microfluidic chamber that has at least one feeder for biomonomers and at least one removal device spatially separated from the feeder.

28. The method of claim 20, wherein the biomonomers are charged and the partial regions of the matrix are selected by applying at least one electric field.

29. The method of claim 20, further comprising:
(f) reacting the synthesized biopolymers with test specimens; and
(g) detecting interactions by a (bio)chemical reaction, including luminescence, radioactive and/or non-radioactive labeling, or biotinylization reactions with streptavidin.

30. (Cancelled)

31. (Cancelled)
